(19) United States
(12) Patent Application Publication
(10) Pub. No.: US 2006/0142680 A1
(43) Pub. Date: Jun. 29, 2006

(54) Active assist for the ankle, knee and other human joints

(76) Inventor: Michael Anthony Iarocci, Patchogue, NY (US)

Correspondence Address: Michael A Iarocci, 64 Woodward Avenue, Patchogue, NY, 11772, US

(21) Appl. No.: 11/025,465

(22) Filed: Dec. 29, 2004

(51) Int. Cl. A61F 5/00

(52) U.S. Cl. 602/16; 602/5; 602/20; 602/3; 602/26

(57) ABSTRACT

A human joint assist device that applies a torque at the joint to assist physiological exertion forces, that is the load carrying task of the joint and surrounding muscles, tendons, and ligaments. The application of this device reduces the physiological exertion force requirement, and may be adjusted with respect to assist level, to suit the issue associated with joint motion and is useful for joint rehabilitation and sports activities. Among other things, this results in a reduction of physiological exertion force in a fashion that makes it easier to extend the levers (long bones) associated with extension against a given resistance. For example, standing from a squatted position with the assist of this device reduces the stress on physiological members associated with joint articulation.

### CROSS-REFERENCE TO RELATED APPLICATIONS

None

### BACKGROUND

1. Technical Field

The present disclosure relates to human joint support systems. More particularly, the present disclosure relates to a device that supports human joints and adds the additional feature of providing torque at the fulcrum point to assist or compliment motion.

2. Background of Related Art

The knee joint is often described as the largest and most complex joint in the human body. The knee joint is the fulcrum of the body's longest lever and is subjected to tremendous bending moments and loads during athletic activity. The ankle joint is also a fulcrum, of the hinge-type and diathrotic (freely moveable), subjected to large bending moment forces. As a result, these vulnerable joints are sites of many injuries. As a one-axial joint, movements of the knee are primarily restricted to flexion and extension; the fully extended knee corresponds to a straight leg where the angle measured on the back side of the leg between the calf and hamstring muscles is approximately 180 degrees, shown as angle α in FIG. 1. Full flexion for a person ordinarily lies somewhere between 10 and 50 degrees (the specific angle for a person is a function of the person's physiology and is limited by contact between the person's calf and thigh or other physiological limitation such as may be caused by a knee injury). Full extension corresponds to a equal to approximately 180 degrees, namely, a straight leg. (Although a straight leg is referred to as having an angle of 180 degrees, this is for ease of reference only. The actual angle for a straight leg is up to a maximum of 180 degrees, but may be slightly more or less depending on an individual's physiology.) In addition to flexion and extension, some rotation of the knee is also possible, especially when the joint is flexed. The ankle joint is similar with the addition of some degree of bending freedom in multiple axes.

The bending moment exerted at these and other joints are a result of muscle contraction in conjunction with a translation and tension of connective tendons, typical of third class physiological lever systems. Using extension of the knee as an example, the main muscle of the extensor group is the quadriceps, which comprises the rectus femoris, vastus medialis, vastus lateralis, and vastus intermedius muscles. All four muscles converge into a common tendon that attaches to the patella (knee cap) and then extends downward and inserts into the tibial (major lower leg bone) tuberosity. The tension applied via this system results in an application of torque at the joint, which tends to straighten (extend) the leg from a flexed position around the knee. Due to the geometric configuration of the joint, a compressive load is also applied to the joint during the period of motion. This is typical for all joints as they are in motion under load in the flexion or extension positions. The compressive load during joint motion is in addition to a compressive load along the primary axis of the joint from weight bearing (for example knee, ankle, or vertebrae compression during standing, or impact compression when running).

FIG. 1 is now used to describe this physiological movement further. In FIG. 1, a portion of a person's leg 10 from the thigh 12 above the knee 14 to below the knee 16 is shown. (Also shown in FIG. 1 is a simple prior art brace, described in more detail immediately below; thus, the leg 10, shown positioned in brace, is shown in outline using dashed lines, and can be “seen through” to reveal the underlying brace structure.) The knee 14 is shown partially bent in FIG. 1. In somewhat simplistic terms, driving the body forward or upwards during the act of walking, running, or stepping is roughly imparted by straightening out the knee 14 shown in FIG. 1. With the knee 14 bent, the quad muscle (above the knee 14 in the thigh 12) contracts and puts the connective tissue with the lower leg bone in tension as described above. Such forces are exerted approximately along a curve that runs from the thigh 12 through the knee 14 and into the lower leg portion 16. Such physiological forces thus supply a torque about the knee pivot axis (shown to coincide with axis A-A) that results in a distributed force that rotates the portion of the leg above the knee (thigh portion 12) counterclockwise and/or the lower leg portion 16 (calf, shin, foot portion) clockwise about axis A-A. This results in extension of the leg, or an increase in angle α to 180°.

Injury occurs to the connective tissue and/or muscles if the tension therein exceeds the elastic limit of the tissue or muscle. This can occur, for example, when the tension supplied by the muscle is too great. The greatest load on these body parts typically occurs when the body is at rest and the muscles must overcome the body's inertia in order to begin running, climbing stairs, jumping, etc. (Sudden impacts to the muscles and/or connective tissue (such as unexpectedly stepping off a curb or into a hole) can also result in ruptures or injuries.)

In addition, certain prior art braces used, for example, in rehabilitating various injuries, are known. FIG. 1 also shows a perspective view of such a prior art device 50 for the knee 14, for example. FIG. 1 shows the portion of the person's leg 10 positioned in the device 50. The brace 50 includes two support members 60l, 60r that lie on the left and right sides, respectively, of the person's leg 10, as shown in FIG. 1. Each support member 60l, 60r has upper and lower portions connected at hinges 130l, 130r, respectively, that allow the upper and lower portions to also pivot about axis A-A. (That is, the device 50 is positioned such that the axis of rotation of hinges 130l, 130r is co-linear with knee axis, both of which thus coincide with axis A-A.)

Thus, left support member 60l is comprised of left upper support portion 60lu and left lower support portion 60ll connected by left hinge 130l. Likewise, right support member 60r is comprised of right upper support member 60ru and right lower support member 60rl connected by right hinge 130r. (Hinges 130l, 130r may each be comprised of a hinge pin that passes through the respective upper and lower portions, having its head welded to one portion and the opposite end capped.) Rigid connecting cradle members 80a, 80b surround the rear of thigh 12, and also attach left and right upper support members 60lu, 60ru. Two corresponding straps 82a, 82b surround the front of the thigh 12 opposite members 80a, 80b and, when tightened, serve to secure the upper portions of support members 60l, 60r to the person's thigh 12 above the knee. (For clarity, conventional aspects regarding cradle members and straps are omitted from the figures, such as their connection to support members, buckles and the like.) In like manner, the combination of rigid connecting cradle members 80c, 80d and corresponding straps 82c, 82c serve to secure the lower portions of support members 60l, 60r to the person's leg below the knee (i.e., to lower leg region 16). Straps 82a-d and cradle members 80a-d so affixed to leg 10 also serve to hold the pivot axis of hinges 130l, 130r co-linear with the pivot axis of the knee 14, namely such that both knee and hinge axes are coincident with A-A in FIG. 1. Thus, the device 50 allows the knee 14 to pivot about axis A-A (as is normal for the knee), while restraining other motion, e.g., translation of the knee along the A-A axis, twisting about the knee joint, etc.

Thus, devices such as that shown in FIG. 1 are useful in restraining certain movements. Other like devices exist for the ankle and other joints. Still other devices can restrict the range of motion in the permitted direction (such as about the A-A axis in FIG. 1). In such devices, the hinges may include mechanisms (stops) that set the range through which upper and lower portions can pivot about the axis. This is also used, for example, when a full range of motion of the joint in early stages of rehabilitation can damage a surgical repair of a tendon, ligament, etc.

Such devices, however, providing support and restraint, are passive. They impart no energy in support of the permitted motion. In other words, the person's body supplies all of the forces that create the pivot about the joint in the permitted direction. In the case of the knee of FIG. 1, the person supplies all of the energy from his muscles for walking, going up stairs, etc., using the quad and other muscles and connective tissue to create a torque about axis A-A as described above. No energy is imparted from the device itself, which only serves to restrain unwanted movement. Thus, the devices do not serve to reduce the force on the muscles, tendons, etc. created by such activity and which can result in injury. Especially in a rehabilitation case, these forces can result in injury to the tendon, ligament and/or muscle, as described above.

### SUMMARY

A technique to guide and support joints, as described in more detail below, includes the application of a local concentrated torque located about the axis of rotation.

An object of the present disclosure comprises applying an external mechanical torque at certain human body joints in assistance of (or complete replacement of) the torque applied physiologically. This reduces the requirement of the physiological torque, thus reducing the risk of injury, including re-injury in a rehabilitation of an injured tendon, ligament or muscle.

Another object of the present disclosure comprises assisting with extension and flexion of joints in a way that relieves some of the stress on the surrounding muscles, tendons, and ligaments associated or adjacent to the particular joint.

Another object of the present disclosure comprises relieving some of the joint compression associated with normal extension and flexion of joints.

Another object of the present disclosure comprises allowing the assist applied by a device to be adjustable and progressive in that as the angle of flexion changes, the torque applied increases in a linear or non-linear manner according to application.

Another object of the present disclosure comprises assisting with joint rehabilitation therapy in that a device allows motion with reduced tensional and torque related load, and to assist healthy joints in sports and other activity.

The present disclosure comprises a human joint assist device having the ability to apply a torque at the joint in question to assist the load carrying task of the joint and surrounding muscles, tendons, and ligaments. The application of this device reduces the load, and may be adjusted with respect to assist level, to suit the issue associated with joint motion and is useful for joint rehabilitation and sports activities.

Among other things, the present disclosure comprises a device for assisting the human body in pivoting about a pivot region (such as a joint) of the human body. In one exemplary embodiment, the device includes a first rigid member that may be affixed to a first region of the human body adjacent the pivot region. It further includes a second rigid member that may be affixed to a second region of the human body adjacent the pivot region. At least one hinge connects the first rigid member and the second rigid member. The device is configured such that hinge mechanism lies adjacent to or in the pivot region when the first rigid member is affixed to the first region of the human body and the second rigid member is affixed to the second region of the human body. In addition, first rigid member rotates with respect to second rigid member about hinge when the first region of the body pivots via pivot region about the second region of the human body.

An assist member, such as a spiral spring, or a helical spring configured/implemented to generate torsional forces, has a first segment attached to the first rigid member and a second segment attached to the second rigid member. Energy is stored in the assist member as the first rigid member is pivoted with respect to second rigid member through hinge in one direction. Energy so stored in the assist member may be released to at least assist pivoting of the first rigid member with respect to the second rigid member about hinge in the opposite direction. When the device is affixed to the first and second regions of the human body, the release of energy from the assist member assists the body in returning the first region and second region back to an initial position.

The assist member (such as a spring) may be a linear or non-linear energy storage device. Where the assist member is substantially linear, the energy stored is linearly proportional to the amount the member is pivoted. In the non-linear case, for example, the amount of energy stored per increment of pivot may change as a function of the pivot. In general, it is desirable that the assist member has little or no energy stored (and thus applies little or no torque or force) when the first and second members of the device correspond to a rest or stable position of the pivot region of the human body. For example, where the device is for assist of the knee and the assist member is a spring, the spring is substantially at rest (decompressed) when the leg is straight.

### BRIEF DESCRIPTION OF THE DRAWINGS

The present invention is described herein with reference to the accompanying drawings, wherein:

FIG. 1 is an example of a brace for the knee as known in the art;

FIG. 2 is an exemplary embodiment showing an assist brace for the knee in accordance with the present invention;

FIG. 2A is a more detailed view of certain components of FIG. 2;

FIG. 3 is a front view of the assist brace of FIG. 2;

FIG. 4 is a more detailed side view of a portion of the device of FIG. 2, including an alternative spiral spring component; and

FIG. 5 is a side view of an alternative exemplary embodiment of the invention.

### DETAILED DESCRIPTION OF PREFFERED EMBODIMENTS

Embodiments of the presently described assist brace will now be described with reference to the accompanying drawings, in which like reference numbers designate identical or corresponding elements in each of the views.

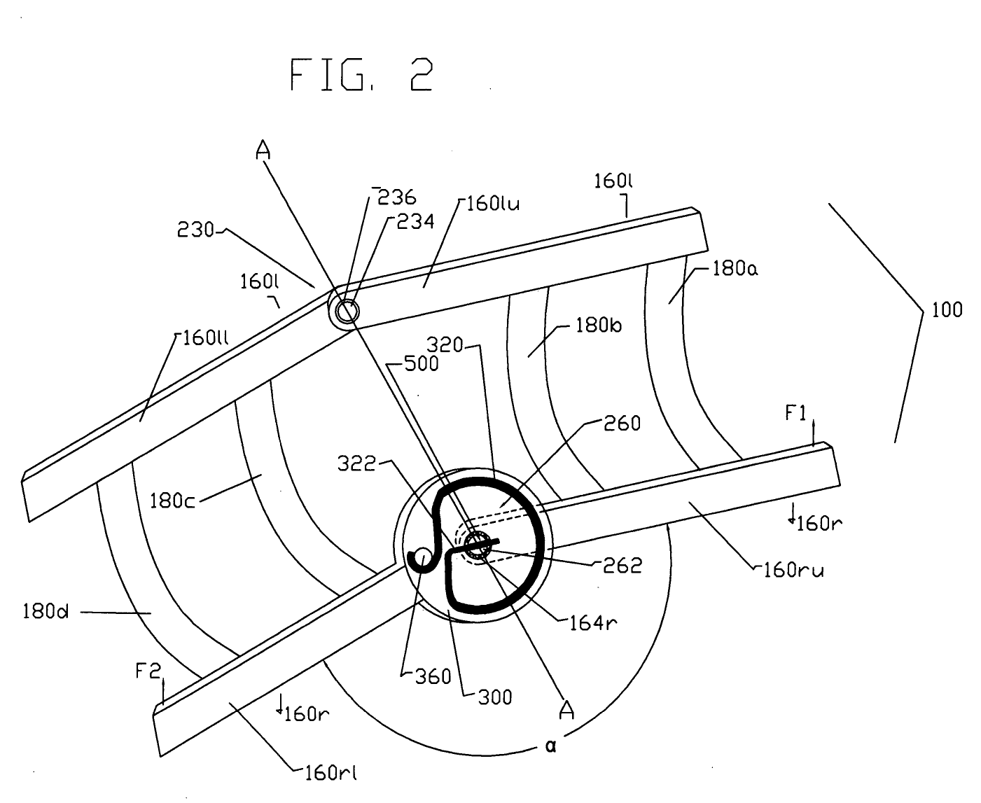

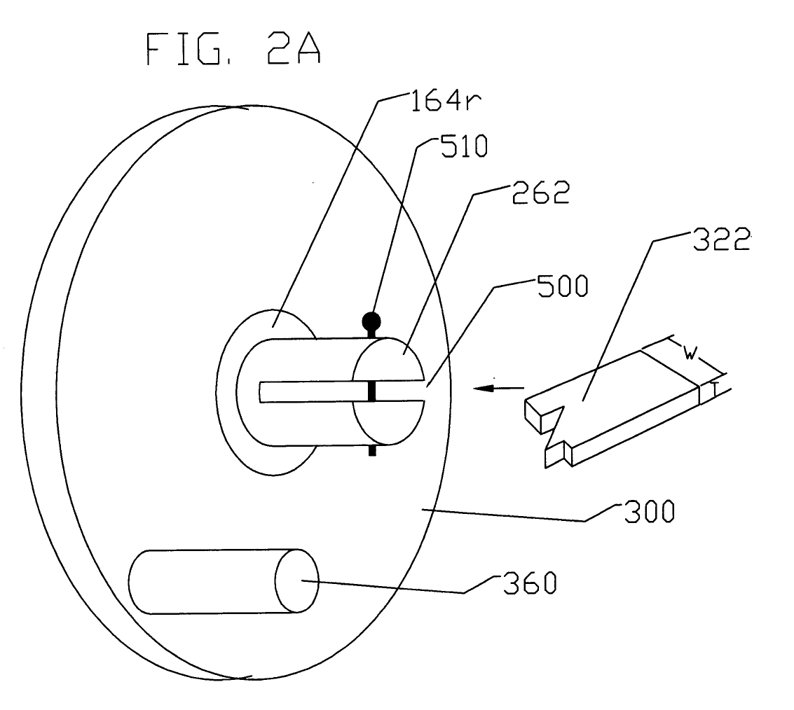

With initial reference to FIG. 2 an exemplary embodiment of an assist brace in accordance with the present disclosure is shown and generally designated as 100. The assist brace 100 in the exemplary embodiment is for the knee, although as described below, the invention can be applied in a device for any joint.

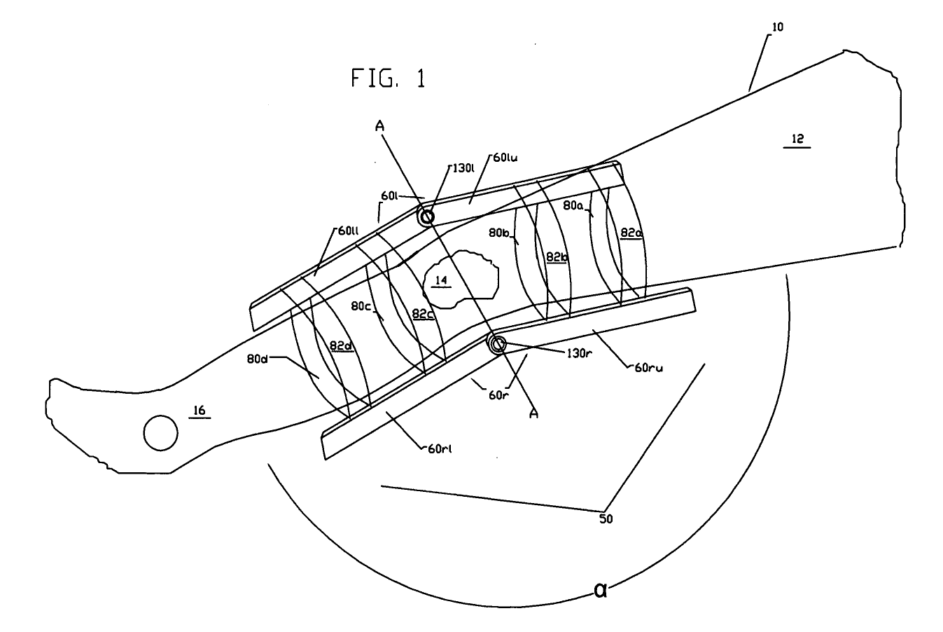

Assist brace 100 includes two support members 160l, 160r that lie on the left and right sides of the person's leg, respectively, as shown in FIG. 2. (Although not shown in FIG. 2, the person's leg would be situated in device 100 in the same manner as the leg 10 is situated in the brace 50 in FIG. 1, namely, with the axis of the knee joint aligned with axis A-A.) Left support member 160l is comprised of left upper support portion 160lu and left lower support portion 160ll connected at hinge 230. Right support member 160r is comprised of right upper support portion 160ru and right lower support portion 160rl connected at hinge 260. Hinges 230, 260 allow the upper portions of left and right support members 160l, 160r to pivot about axis A-A with respect to the respective lower portions. When mounted on a person's leg, rigid connecting cradle members 180a, 180b that lie behind the person's thigh and corresponding straps (not shown, but like those shown in FIG. 1) that surround the front of the person's thigh secure upper portions of left and right support members to the person's leg above the knee. Likewise, rigid connecting cradle members 180c, 180d that lie behind the person's calf and corresponding straps (not shown) secure lower portions of left and right support members below the knee. Thus, device 100 of the exemplary embodiment allows the knee to pivot about axis A-A.

The assist brace of FIG. 2 includes components for generating an external assist in the form of a restoring torque about axis A-A that supplements the physiological forces that serve to straighten the leg and which generates the forward/upward driving force needed for walking, running, jumping, etc. Hinge 230 as shown in FIG. 2 may be a traditional hinge. However, hinge portion 260 of FIG. 2 interfaces with additional components that generate the external assist.

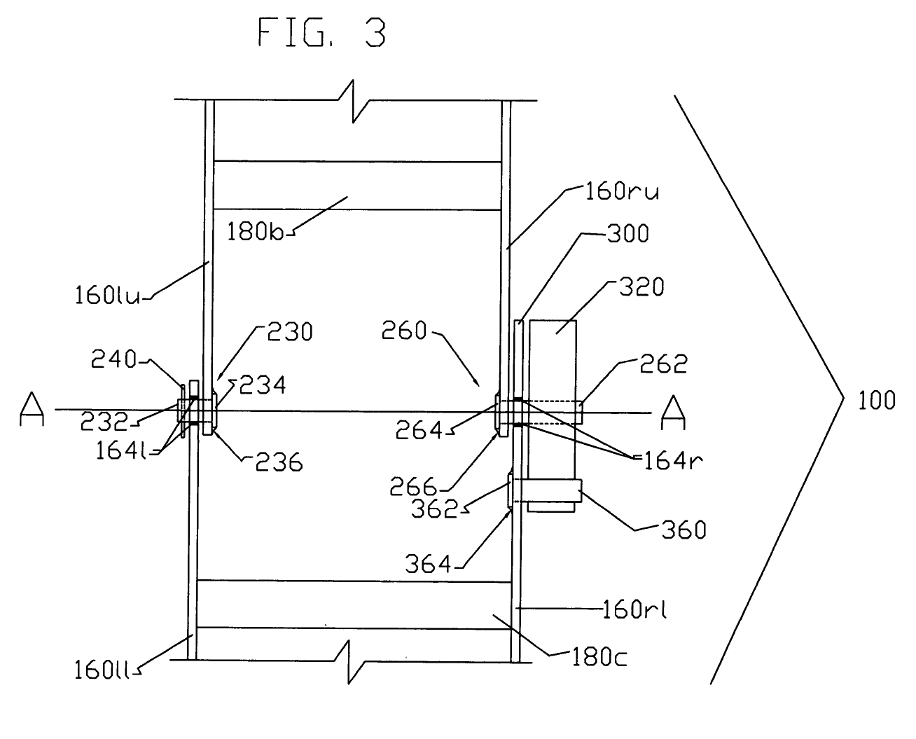

It is first noted that, generally, it is preferable for the assist structure to lie on the outer side of the body, to avoid interference with the other leg. Thus, brace 100 shown in FIGS. 2 and 3 will preferably be for the left knee. For the right knee, active structure would be on the outer side of left support member 160l.

Focusing on FIGS. 2 and 3 together, the standard hinge 230 is first described. In FIG. 3 (a front view of the assist brace 100 of FIG. 2), hinge 230 is shown to the left. As seen, hinge 230 is comprised of a pin 232 having a contiguous head 234 that is welded 236 (and thus affixed) to upper portion 160/u of left support member 1601. Pin 232 extends through left upper portion 160lu and also through bearing 164l in left lower portion 160ll. (The projected portion of bearing 164l is shown in FIG. 3.) Left lower support portion 160ll thus rotates freely about pin 232 at bearing 164l, while cap 240 affixed to pin 232 on the outer side of left lower support portion 160ll retains left lower support portion 160ll on pin 232. Thus, hinge 230 allows left upper and lower portions 160lu, 160ll of left support member 160l to rotate with respect to each other about axis A-A.

Hinge 260 for right support member 160r interfaces with additional assist structure. It is first noted that the top of right lower support portion 160rl includes circular spiral spring support plate 300 centered at axis A-A and lying perpendicular to axis A-A. Hinge 260 likewise has a pin 262 having head 264 welded 266 to inner surface of right upper support portion 160ru, and which passes through bearing 164r in plate 300 of right lower support portion 160rl, thus also allowing right upper and lower portions 160ru, 160rl to rotate with respect to each other about axis A-A. Pin 262 is longer than pin 232 and extends further to the outer side of right lower support portion 160rl. (The full extent of pin 232 along axis A-A is shown in FIG. 3, using dashed lines when it lies behind spring 320 and other components.)

As best seen in FIG. 2, spiral spring 320 is positioned on the outer side of hinge 260 and adjacent support plate 300 such that the plane of spring 320 lies parallel to the plane of right support portion 160r, including lying parallel to support plate 300. The center of spring 320 is aligned with axis A-A. Bent segment 322 at the center of spring 320 is bent perpendicular to axis A-A and extends perpendicular to axis A-A through slot 500 in pin 262. (A closer view of the details of this portion of FIG. 2 is shown in FIG. 2A, which also reveals the flat band-like cross section of the spiral spring 320 (that is, having a relatively large width W and relatively small thickness T.)) Bent segment 322 is held in slot 500 by a restraining device that prevents the spring from sliding out of the slot. FIG. 2A shows an example of such restraining device being a cotter pin 510 that passes through a bore in pin 262 near the face of pin 262 and perpendicular to its axis. It is noted that retaining the bent segment 322 serves to hold spring 320 in place adjacent support plate 300. Retaining bent segment 322 in slot 500 serves to effectively attach center of spiral spring 320 with respect to pin 262 and thus the right upper support portion 160ru.

The opposite (outer) end of spring 320 is contoured such that it hooks about second pin 360 that extends from support plate 300 near its circumference below axis A-A. (Second pin 360 includes head 362 that is welded 364 to support plate 300, as seen in FIG. 3. Thus, the opposite end of spiral spring 320 is in mechanical contact with right lower support portion 160rl.) Outer end of spring 320 may completely encircle second pin 360, thus also serving to retain spring 320 in place and to attach spring 320 to right lower support portion 160rl.

It is noted that the basic geometry of the spring 320 plane lying against pin 360 and pin 262 tends to produce frictional restraint to hold spring 320 in place, without the need to unduly restrain or affix the ends of spring 320 with respect to pins 262, 360. Thus, as shown in FIGS. 2 and 2A, spring 320 will stay in place using the simple cotter pin 510 through slot 500. This effectively serves to hold spring 320 in place and adjacent support plate 300 even when the spring 320 is non-compressed, without the need to affix the hook of the outer end of spring 320 against second pin 360. In addition, a hook configuration as shown in FIG. 2 will naturally stay in contact with second pin 360 due to the spring forces as alpha decreases below 180°, thus effectively attaching outer end of spring 320 to right lower support portion 160rl. This also provides easier replacement of spring 320.

Spring 320 is configured and pin 360 is positioned such that when the right upper and lower support portions 160ru, 160rl lie substantially in a straight line (i.e., when a leg inserted in device 100 is straight), the spring is in a substantially non-compressed state. As shown in FIG. 2, spring 320 is positioned such that when the knee is bent spring 320 is compressed about axis A-A. That is, right upper support portion 160ru is rotated clockwise about axis A-A with respect to right lower support portion 160rl such that a decreases below 180°. Compression of spring 320 consequently gives rise to a force on upper and lower support portions 160ru, 160rl to straighten out the leg (i.e., to return a to 180°).

Thus, as the knee is bent (corresponding to a decreasing below 180°), for example, when squatting in preparation to jump, energy is stored in the spring. Such storing of energy in the spring generally does not come at a significant physiological cost, since it typically corresponds to either a capture of gravitational potential energy through a lowering of the center of gravity of the body, and/or a partial capture of kinetic energy when walking or running. As the leg begins to straighten (i.e., as a increases back to 180°), for example, in executing the jump, the energy of the spring is released in aid of the jump. Thus, the energy of the spring assists the muscles, tendons and ligaments in execution of the jump, thereby reducing the stress and risk of injury. Such assist will also occur for more static situations. For example, when a person squats down, like a baseball catcher, the energy is likewise stored in the spring. When the person subsequently stands up, the energy stored in the spring is released to aid in the straightening of the leg.

In more technical terms, referring again to FIGS. 2 and 3, as the person flexes his or her leg from the straight position to a bent position about the knee (a decreasing below 180°), left upper support portion 160lu pivots with respect to left lower support portion 160ll on hinge pin 232 about axis A-A, and right upper support portion 160ru pivots with respect to right lower support portion 160rl on hinge pin 262 on active hinge side about axis A-A. As noted above, the outer end of spring 320 is effectively secured to plate 300 of right lower portion 160rl (via second pin 360) and inner end 322 of spring 320 is effectively secured to right upper support portion 160ru (via hinge pin 262); rotation of upper and lower support portions in the direction of decreasing a imparts a rotational compression of the spring about axis A-A, which results in a buildup of strain energy in spring 320. For one such exemplary spring, the energy stored in spring 320 increases approximately linearly with a decrease in the angle α. The angle alpha is largest when standing erect and smallest when the knee is bent to the maximum angle, as when in the squatted position.

It is again noted that, from a physiological standpoint, reducing the angle alpha corresponds to bending the knee. This is generally accomplished without significant energy expended by the muscular-skeletal system, since it is principally gravity assisted. For example, bending the knee to begin running (corresponding to decreasing a below 180°) is largely a controlled lowering of the torso of the body on the leg. Thus, for such activities the spring 320 principally captures energy from the person's body weight (gravitational potential energy), not physiological energy.

Subsequent straightening of the leg (corresponding to increasing α back to 180°) typically corresponds to a substantial expenditure of physiological forces and stresses, since it is the driving of the body weight for a jump, the driving force for beginning or continuing running or walking, etc. In the case of a jump, initiation of running, or other movement from rest to motion, the physiological forces are typically greatest in order to overcome the body's inertia. For this movement (restoring a back to 180°), the stored energy in the coiled spring 320 is transferred back, as a torque about axis A-A in the direction of increasing α, thus assisting in straightening the leg. This torque is applied to right upper and lower support portions 160ru, 160rl via hinge pin 262 and second pin 360, respectively.

Thus, the torque applied from bent segment 322 of spring 320 to slot 500 of hinge pin 262 is transferred to weld 266 (see FIG. 3) that results in a distributed force in right upper support portion 160ru in the direction of increasing a (represented in FIG. 2 as force F1). With a leg inserted in device 100, this force in turn is distributed to the thigh via the right upper support portion 160ru, rigid connecting cradle members 180a, 180b surrounding the back of the thigh above the knee inserted therein, and left upper support portion 160lu which flanks the opposite side of thigh. (The corresponding straps (not shown) surrounding the front of thigh opposite cradle members 180a, 180b serve to hold device 100 in place with respect to an inserted leg in conjunction with cradle members 180a, 180b). Again, as noted, the force acts to assist straightening of the leg, i.e., in the direction of increasing a back to 180°.

Similarly, a distributed force is applied by the outer portion of compressed spring 320 to second pin 360, which is transferred to spring support plate 300, then onto contiguous right lower support portion 160rl. This results in a distributed force in right lower portion 160rl also in the direction of increasing a (represented in FIG. 2 as force F2). This force in turn is distributed to the leg below the knee via the right lower support portion 160rl, connecting cradle members 180c, 180d surrounding the rear of calf and shin inserted in device 100, and left lower support portion 160ll which flanks the opposite side of leg below the knee. (The corresponding straps (not shown) surrounding the front of shin opposite cradle members 180c, 180d serve to hold device 100 in place in conjunction with cradle members 180c, 180d). Thus, the forces arising from compressed spring 320 both above and below the knee axis A-A act to assist straightening of the leg, i.e., in the direction of increasing a back to 180°. The distributed forces in embodiments above in the release of energy from the spring results in a reduction of the physiological force requirement during straightening of the leg or other joint the assist brace is applied to. (Considering the elementary physics of the device, the magnitudes of F1 and F2 are equal, since they both arise from the compressed spring.)

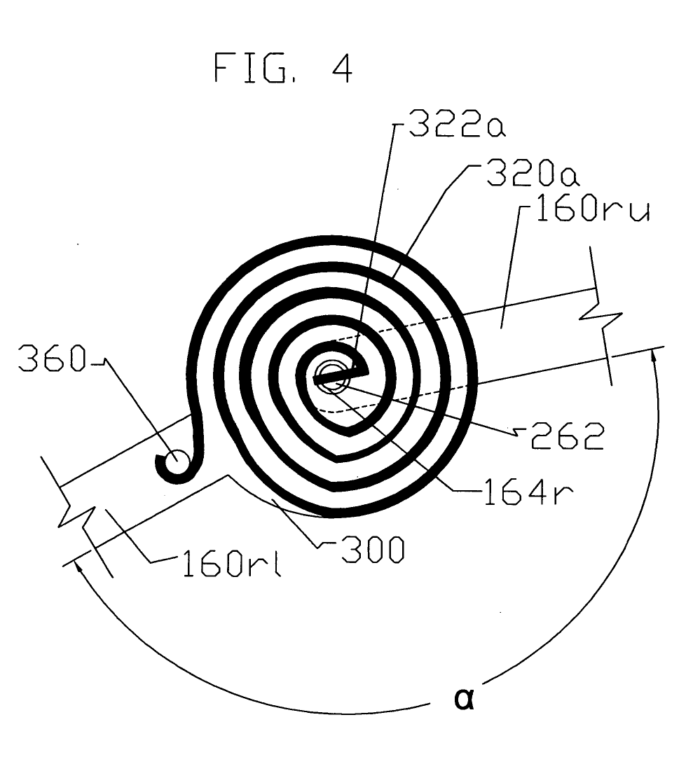

FIG. 4 shows a more detailed right side view that focuses on the active hinge portion and adjacent structure of the right side of device 100 of FIGS. 2 and 3. It is noted that in FIG. 4 the spring has additional spiral turns (and is thus designated with ref no. 320a). Additional turns, as in FIG. 4, allow the thickness and width to increase. This demonstrates that the spring (and the spring constant) may be fitted for the specific need of the user. For example, if the user is in the initial stages of rehabilitation from a surgically repaired ACL, the spring constant may be relatively high so that the stress on the ACL in walking, climbing stairs etc. is reduced.

One skilled in the art may readily determine a spring configuration for a given situation. For example, in the case of a 180-pound male, the normal physiological torque applied at the knee joint for straightening the leg when in a full squat position or full flexion (taken as alpha=30° for this example) is approximately 125 ft-lbs per knee. If half of this straightening force is to be applied physiologically and half by the assist mechanism is desired at the position of full flexion, an applied torque of approximately 63 ft-lbs per knee may be applied via the “active assist brace.” One skilled in the art can readily calculate that a spiral spring suitable for this example has overall dimensions of approximately 3 inches outer diameter and 1-inch inner diameter, while the cross-sectional dimension of the spiral spring are approximately 1-inch width and 3/16 inch thickness. As noted, this spring will apply approximately 63 ft-lbs of torque when compressed 150° (i.e., from 180° to 30°). At an intermediate squat position, corresponding to alpha=110°, the spring torque in this example is approximately 30 ft-lbs or about equal to 40% of the physiological torque of approximately 75 ft-lbs. The assist torque at the straight leg position is 0 ft-lbs. In the above example if full assist is desired at alpha=30°, a suitable spring has width of 1.5 inches with all other spring parameters remaining the same. The above example utilizes a spring with a substantially linear torque/rotation relationship; nonlinear spring force may alternatively be used so to favor specific torque application at specific deflection angles. Spring rates, maximum torque, and torque/rotation profile may be readily calculated and a suitable spring devised based upon user weight, physiological condition, desired assist, among other factors.

It is also noted that the assist does not have to begin and end with a straight leg, that is, spring 320 of FIGS. 2-3 does not have to engage immediately as the person bends his or her leg. For example, pin 360 may be positioned on plate 300 such that it does not engage the hook of the outer end of spring 320 (and thus spring 320 does not begin to compress) until alpha is less than or equal to 170°. (Thus, plate 300 may have a number of second pins 360 at various positions, or may have holes at various positions in which second pin 360 may be placed.)

While the invention has been described with reference to several embodiments, it will be understood by those skilled in the art that the invention is not limited to the specific forms shown and described. Thus, various changes in form and details may be made therein without departing from the spirit and scope of the invention. For example, the assist brace may be readily adapted to other joints, including the ankle and shoulder. It may also be adapted to the back, so as to assist a person who is bent over in standing up straight. In addition, assist springs may be located on both the left and right hinges.

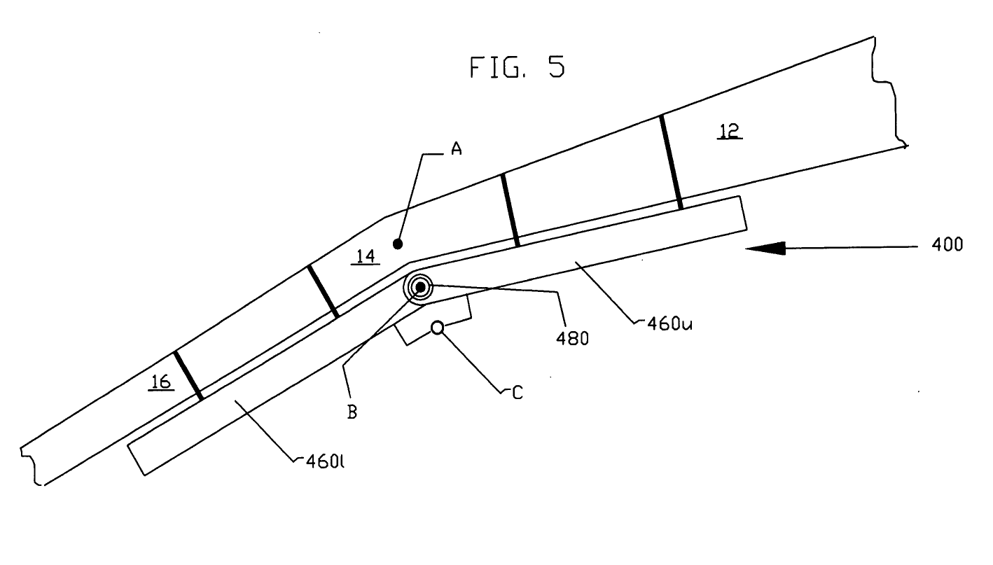

In addition, the assist device does not have to pivot coincident with the axis of the joint. The pivot axis of the assist device may lie substantially parallel, but separate from, the pivot axis of the joint. Referring to FIG. 5, for example, a single support member 400 may be adapted to strap behind the leg above and below the knee. Upper support portion 460u is strapped to the backside of thigh and lower support portion 460l is strapped behind the shin and calf Upper and lower support portions 460u, 460l are attached by a hinge 480 which rotates about axis B (perpendicular to the plane of the figure) when the knee pivots about its axis A (also perpendicular to the plane of the figure). Adjacent to hinge 480 are active-assist components, generally represented by element C, which may be a spring that interfaces with upper and lower portions analogous to the above embodiments and properly shaped for the position behind the leg. The spring has central (torsional) axis aligned with axis B, and stores energy as knee is bent about axis A. (Spring may be helical with the direction along the helix parallel to axis A-A positioned against the back of the knee having a slight arc that bends and contours at least in part to a flex of the knee.) As the leg is straightened, the stored energy is released by the spring as a distributed force to upper and lower members 460u, 460l in assistance of straightening the leg.

It is noted that in the embodiment of FIG. 5, as the knee is bent, the geometry may tend to create a shear motion between the upper member 460u and the leg, and between the lower member 460l and the leg. With the upper and lower members 460u, 460l strapped to the leg, this tendency to slide across the skin may result in irritation or discomfort. Thus, a standard shear plane may be incorporated into the upper or lower members 460u, 460l near the hinged joint so not to transfer the shear load to the skin. It is further noted that a device for the spine is more suitable to attachment to a person's back, rather than extending up and down the sides of the person (where the pivot axis or axes of the spine and device may be made substantially co-axial). With placement of the device on the back, the pivot axis (or axes) of the vertebra and the pivot axis (or axes) of the device can be made parallel but not coincident. (This is analogous to the separation between axes as shown for the knee in FIG. 5). With placement of a device on the back, a series of springs may be placed on the device corresponding to one or more vertebra from top to bottom of the spine (or some portion of the spine). Although the springs would not be coincident with the pivot axes of the vertebra, there would be much less shear because of smaller excursion angle between vertebra. Thus, a shear plane may not be necessary for certain configurations.

In addition, it is clear, for example, that upper and lower support members are not limited to those shown in the exemplary embodiments above, which are essentially standard knee brace segments. As readily understood by one skilled in the art, they may be designed or configured in many different ways for use in the assist provided by the device. For example, the support members may also (or alternatively) comprise supportive sleeves, which may be non-rigid (e.g., neoprene) or partially or completely rigid (e.g., plastic). As another example, a pneumatic cylinder may be substituted as the assist mechanism. As yet another example, in certain configurations and applications, it is not necessary to have a hinge attaching upper and lower support members on the active side. A hinge may be omitted, for example, in cases where the spring has good axial stability and support portions having extensive and close contact with the respective body portions. In that case, the spring may have one end attached directly to the upper support portion and the other end attached directly to the lower support portion.

Thus, the particular devices and techniques described above are by way of example only and not to limit the scope of the invention.

What is claimed is:

1. A device for assisting the human body in pivoting about a pivot axis of the human body, the device comprising:
a) a first rigid support member affixable to a first region of the human body adjacent to the pivot axis of the body;
b) a second rigid support member affixable to a second region of the human body adjacent to the pivot axis of the body;
c) at least one hinge connecting the first rigid support member and the second rigid support member, a pivot axis of the hinge lying substantially parallel to the pivot axis of the body when the first rigid support member is affixed to the first region of the body and the second rigid support member is affixed to the second region of the body; and
d) an assist member having a first segment coupled to the first rigid support member and a second segment coupled to the second rigid support member,
wherein the assist member stores energy when the first support member is pivoted with respect to the second support member in one direction about the axis of the hinge, and the energy stored in the assist member is releasable to pivot the first rigid support member with respect to the second rigid support member about the axis of the hinge in the opposite direction.

2. The device as in claim 1, wherein the assist member is a spring.

3. The device as in claim 2, wherein the spring is one of a spiral spring, helical spring and pneumatic cylinder.

4. The device as in claim 1, wherein the first rigid support member comprises a portion of a therapeutic brace that affixes above a human joint and the second rigid support member comprises a portion of a therapeutic brace that affixes below the joint.

5. The device as in claim 4, wherein the therapeutic brace is a knee brace, the pivot axis of the body corresponds to the bending axis of the knee, the first rigid support member attaches to the thigh region and the second rigid support member attaches to the calf region.

6. The device as in claim 5, wherein the assist member stores energy when the knee is bent and releases energy when the knee is straightened.

7. The device as in claim 4, wherein the therapeutic brace is an ankle brace, the pivot axis of the body corresponds to the axis of dorsiflexion and plantar flexion of the ankle, the first rigid support member attaches to the shin and the second rigid support member attaches to the foot.

8. The device as in claim 7, wherein the assist member stores energy when the ankle is dorsiflexed and releases energy as the ankle is plantar flexed.

9. The device as in claim 4, wherein the assist member is a spring.

10. The device as in claim 1, wherein the first rigid support member comprises a portion of a therapeutic back brace that affixes to a person above one or more select vertebra and the second rigid support member comprises a portion of a therapeutic back brace that affixes to the person below the one or more select vertebra, the pivot axis of the body comprising at least one of the pivot axes of the one or more select vertebra.

11. The device as in claim 1, wherein the pivot axis of the human body is substantially coincident with the pivot axis of the hinge when the device is affixed to the body.

12. The device as in claim 1, wherein the assist member is directly attached to at least one of the first rigid support member and the second rigid support member.

13. The device as in claim 1, wherein the assist member is indirectly attached to at least one of the first rigid support member and the second rigid support member.

14. The device as in claim 1, wherein the energy stored in the assist member when released provides a torque on the first and second support members, the torque acting to pivot the first rigid support member with respect to the second rigid support member about the axis of the hinge in the opposite direction.

15. The device as in claim 14, wherein the torque provided by the released energy reduces a physiological force needed to pivot the first region of the human body with respect to the second region of the human body about the pivot axis of the human body when the first rigid support member is attached to the first region of the body and the second rigid support member is attached to the second region of the body.

16. A device for assisting the human body in pivoting about a pivot axis of the human body, the device comprising:
a) a first portion of a brace affixable to a first region of the human body adjacent to the pivot axis of the body;
b) a second portion of a brace affixable to a second region of the human body adjacent to the pivot axis of the body;
c) at least one joint between the first brace and the second brace portions, the joint allowing the first brace portion to pivot with respect to the second brace portion, the pivot axis of the joint lying substantially parallel to the pivot axis of the body when the first brace portion is affixed to the first region of the body and the second brace portion is affixed to the second region of the body; and
d) an assist member having a first segment mechanically coupled with the first brace portion and a second segment mechanically coupled with the second brace portion,
wherein the assist member flexes to store energy when the first brace portion is rotated with respect to second brace portion about the axis of the joint from an initial relative position, and the energy stored acts to restore the initial relative position of the first and second brace portions about the axis of the joint.

17. The device as in claim 16, wherein the assist member is a spring.

18. The device as in claim 16, wherein the pivotable joint is a hinge.
